(19) United States
(12) Patent Application Publication
(10) Pub. No.: US 2004/0053829 A1
(43) Pub. Date: Mar. 18, 2004

(54) Fusion protein from antibody cytokine-cytokine inhibitor (selectokine) for use as target-specific prodrug

(76) Inventors: Klaus Pfizenmaier, Tiefenbronn (DE); Thomas Wust, Stuttgart (DE); Dieter Moosmayer, Berlin (DE); Matthias Grell, Darmstadt (DE); Peter Scheurich, Stuttgart (DE)

Correspondence Address: WEINGARTEN, SCHURGIN, GAGNEBIN & LEBOVICI LLP, TEN POST OFFICE SQUARE, BOSTON, MA, 02109, US

(21) Appl. No.: 10/380,438

(22) PCT Filed: Sep. 17, 2001
(86) PCT No.: PCT/EP01/10730

(30) Foreign Application Priority Data
Sep. 15, 2000 (DE) 100 45 592.1

(51) Int. Cl. C07K 14/52; A61K 38/19

(52) U.S. Cl. 514/12; 530/351; 424/85.1

(57) ABSTRACT

The present invention relates to a polypeptide having preferably antitumoral and/or immunomodulating cytokine properties, which can be activated by processing in vivo comprising a central region with specific biological activity. At its C-terminus said region has a region with a processing unit and an inhibitor domain, while at the N-terminus of the central region, there is a region that selectively recognizes a macromolecule on a cell surface or a component of the extracellular matrix.

### SPECIFICATION

The present invention relates to a polypeptide having preferably antitumoral and/or immunomodulating cytokine properties, which can be activated by processing in vivo comprising a central region with specific biological activity. At its C-terminus, said region has a region with a processing unit and an inhibitor domain, while at the N-terminus of the central region, there is a region that selectively recognizes a macromolecule on a cell surface or a component of the extracellular matrix.

Hitherto it has been possible successfully to use recombinant tumor necrosis factor (TNF) and other active substances for the treatment of tumor diseases, for example, only for very limited indications (melanoma/sarcoma metastases of the extremities) under special complicated treatment protocols (e.g. by means of so-called “isolated limb perfusion”), owing to severe systemic side effects that must be regarded as therapy-limiting. From these clinical data it can be estimated that antitumoral efficacy would require a TNF dose about 10 to 100 times higher than the MTD (maximum tolerated dose) than the massive systemic side effects would permit.

Thus the object of the present invention is to avoid or reduce the undesired consequences of a treatment with therapeutically active polypeptide active substances such as TNF-containing substances, while at the same time retaining or even strengthening the therapeutically active, e.g. antitumoral properties of the active substance, such as TNF.

This object is achieved by the embodiments of the present invention characterized in the claims.

In particular, according to the invention a polypeptide with an amino acid sequence is made available that comprises from the N- to the C-terminus

(1) a region that selectively recognizes a specific macromolecule on a cell surface and/or a component of the extracellular matrix,

(2) a region that comprises a peptide linker,

(3) a region with a biological activity for a specific target molecule,

(4) a region that features at least one processing site, and

(5) a region that inhibits the biological activity of region (3) by intramolecular binding and/or interaction,

wherein the biological activity of region (3) can be released by the processing in vivo of the at least one processing site in region (4).

The polypeptide according to the invention, also referred to below as “selectokine,” is an active substance of modular construction, according to a particularly preferred embodiment a preferably homotrimeric fusion protein with a cytokine, preferably TNF or a biologically active derivative or a biologically active mutant thereof, as an antitumoral active substance or region (3), which releases its biological activity by linking with four other function modules specifically in the diseased tissue, e.g. a tumor area. This is achieved by the N-terminus linkage of the therapeutically active substance, e.g. of the TNF molecule, with a targeting module (1) that is specific for the target tissue, e.g. tumor-specific antibodies or derivatives thereof, such as scFv antibody, and by the C-terminus linkage with an inhibitor (5) against the therapeutically active substance, in particular a peptide inhibitor, which is selectively inactivated in the target tissue, such as the tumor area, by processing of the domain (4), preferably by removing from the fusion protein by specific proteolytic cleavage, and thus a bioactive substance bound to the selective targeting module, e.g. TNF, is formed. Between the targeting module (e.g. the scFv antibody fragment) and the module with therapeutic function (e.g. TNF), there is a peptide linker domain (2), preferably a trimerization domain, that ensures the formation of covalent disulfide bridges and thus a regular and stable homotrimerization of the fusion protein.

With the construct according to the invention, it is possible to achieve locally high active concentrations of the therapeutically active substance, e.g. the TNF, without the occurrence of systemically elevated levels of the therapeutic agent (e.g. TNF in the serum) and thus therapy-limiting side effects. At the same time, for example in the case of TNF as a therapeutically active domain, by the targeting module (e.g. antibody) mediated presentation of the inside (in situ) activated TNF, an effect is achieved that corresponds to that of the natural membrane TNF, i.e. it results in the co-activation of both TNF receptor types and thus the potentiation of the antitumoral properties of TNF. By selecting the specificity of the targeting module, a therapeutic agent can be produced that is specifically matched to/optimized for the respective tumor entity.

The (amino acid sequence) regions or modules of the polypeptide according to the invention are described in detail below with reference to preferred embodiments.

The polypeptide according to the invention (selectokine) makes available a novel prodrug technology and is a construct that according to a preferred embodiment comprises a recombinant, homotrimeric fusion protein that in principle comprises a defined sequence of the following structural elements (in the monomer) (N-terminus to C-terminus): (1) a murine, humanized or human single-chain antibody fragment (scFv) of defined antigen specificity consisting of VH-linker-VL; (2) a peptide linker with intrinsic trimerization properties; (3) a TNF molecule that corresponds for example to wild type TNF or to the extracellular domain of the TNF (mature 17 kDa form, M 1-157, Swissprot #P01375) or biologically active variants derived therefrom; (4) a variable linker peptide with specific protease cleavage sites, (5) a specifically TNF-binding protein or peptide.

The targeting module (1) is preferably specific for a cell surface molecule that is expressed in tumor lesions and/or proliferating endothelial cells associated with the process of angiogenesis. According to another preferred embodiment, the targeting module (1) is specific for a component of the extracellular matrix present in tumor lesions and/or angiogenesis areas of pathological lesions. According to a further preferred embodiment, the targeting module (1) is specific for a component of the malignant tumor cell itself. Preferably the region or the module (1) comprises an antibody (e.g. murine, humanized or human) or a fragment thereof, e.g. a Fab fragment or a typical single-chain antibody fragment (scFv) produced according to the prior art, of murine origin, completely human origin, or humanized by CDR grafting, with specificity for an antigen expressed e.g. in the tumor tissue preferably selectively or dominantly, whereby this antigen can be expressed in principle on the malignant cells themselves, but is preferably expressed in the non-malignant portion of the tumor, the stroma cells or the tumor endothelium. Such antigens of non- malignant tissue portions of a solid tumor (carcinoma) are on the one hand genetically invariant, and on the other hand occur in a great variety of tumor entities and are thus universal tumor markers. Reference is made here, for example, to the VEGFR complex or the VEGFR/VEGF complex (as an example of receptor/ligand complexes), as well as to the integrin αvβ3, endosialin, and the fibronectin isoform bFn as selective target structures of the tumor endothelium, and the so-called fibroblast activation protein (FAP) as a selective marker for a component of the extracellular matrix present in the tumor stroma, which can be detected effectively for example with specific, high-affinity scFv. Other examples of suitable targeting modules are peptides, artificial antibodies, and mirror-image nucleic acids (Spiegelmers).

The peptide linker region (2) is preferably a trimerization module and connects the targeting region (1) with the therapeutically active region (3). According to a particularly preferred embodiment, the trimerization module comprises a naturally occurring or synthetic peptide with intrinsic trimerization properties. A particularly suitable example of such a peptide is a domain of the tenascin molecule (AA 110-139, Swissprot #P10039, (chicken) or Swissprot #P24821 (human)). It produces the bond between the targeting module (1) (e.g. scFv) and the therapeutic agent (3) (e.g. TNF) and simultaneously ensures the covalent, homotrimeric linking of the fusion protein during biogenesis.

As explained above, the therapeutically active module (3) preferably contains an amino acid sequence of a cytokine or a therapeutically active fragment thereof. Preferably region (3) contains the amino acid sequence of TNF, more preferably of a TNF precursor protein, and most preferably of a protein identical to the processed, mature wild type TNF molecule (AA 1-157, Swissprot #P01375), or derivatives derived therefrom or mutants with selective receptor binding properties or mutants or derivatives that have been optimized with respect to their specific bioactivity or other properties (stability, protease resistance).

The processing module (4) is for example protease-sensitive (i.e. the processing site corresponds to the recognition sequence of a protease) and preferably its amino acid composition and total length are such that it permits the homotrimerization of the fusion protein effected by the trimerization module and TNF itself, but simultaneously also allows a high-affinity, stable binding of the TNF inhibitor situated at the C-terminus of the molecule (e.g. the extracellular TNF receptor domain) to the TNF moiety, so that the binding of the TNF module to cell-expressed TNF receptors is prevented by this means. Furthermore, the linker is preferably constituted such that it contains at least one, preferably several, selective cleavage sites for those extracellular or cell-associated proteases that are preferably detected selectively in the tumor tissue. Examples of suitable cleavage sites are those for urokinase-type plasminogen activator (uPA), tissue plasminogen activator (tPA), the activated coagulation Factor Vlla, matrix metalloproteases such as MMP-2 and MMP-9, and for the FAP protease expressed membranously with high selectivity in the stroma of tumors. Particularly preferred protease-sensitive cleavage sites are those of matrix metalloproteases associated with the process of metastatic spread and angiogenesis (e.g. MMP-9 recognition sequence Gly-Pro-Leu-Gly-Val-Arg-Gly-Lys; SEQ ID NO 18), of heparanase, of enzymes that occur preferentially in necrotic lesions, and of enzymes associated with prostate cancer (e.g. PSMA, PSA, cleavable processing module glutaryl-(4-hydroxypropyl)-Ala-Ser-cyclohexaglycyl-Gln-Ser-Leu-COOH). The structure of the linker is selected such that the protease recognition sequence is freely accessible, i.e. an effective processing by specific proteases is possible, and after cleavage of the fusion protein, amino acids of the linker that may be remaining on the TNF molecule do not have an adverse effect on the bioactivity of the therapeutically active region.

According to a preferred embodiment of the polypeptide according to the invention, the inhibitor module (5) is a receptor for a cytokine or a fragment thereof. Moreover, the inhibitor module preferably features at least one binding site for the therapeutically active region (3). When TNF is used in region (3), the inhibitor module preferably comprises the complete or partial extracellular domain of a human TNF receptor, e.g. huTNFRl (synonymous with p55/60TNFR; Swissprot #P19438, M 1-190; or fragments of this molecule, for example, M 1-157 or M 60-120). Other proteins binding specifically to TNF, for instance, the extracellular domain of huTNFR2 (EMBL data bank #M32315) or proteins of viral origin such as e.g. the T2 protein, as well as synthetic peptides respectively derived therefrom, which have the ability to bind to TNF and interfere with the TNF binding to cell membrane TNF receptors, are likewise suitable. Due to the binding or interaction of the inhibitor with the therapeutically active module, the fusion protein according to the invention is biologically inactive in this state, i.e. it is in the pro form (prodrug).

The polypeptide according to the invention can include further domains. For example, suitable marking sequences can be added to simplify the purification of the proteins produced by recombination and to simplify in vitro analysis. Thus e.g. a myc-His₆ tag derived from the POPE vector can be added to the C-terminus at region (5), preferably to the TNFR fragment. Further marking sequences are known to those skilled in the art.

The TNF selectokine preferred according to the invention is a covalently linked, homotrimeric molecule consisting of the fusion of three function domains explained in detail above, the tumor-specific antibody module, TNF, and the blocking TNF-binding protein (extracellular receptor domain or peptide derived therefrom), as well as intermediate functional linkers with trimerization properties or specific protease cleavage sites, which in this complete state is inactive as far as TNF activity is concerned. After in vivo administration, the selectokine is first enriched specifically in the tumor area by the antibody moiety and is processed there by the proteases formed by the tumor itself or by the reactive tumor stroma/tumor vascular system (e.g. FAP, uPA, tPA, MMP2, Factor VIIa), i.e. the inhibiting peptide (5) is cleaved off. After selective proteolytic cleavage, the TNFR fragment/inhibitor peptide dissociates from the trimeric TNF molecule, the latter thus becomes bioactive (i.e. the biological activity of the region is released by processing the processing site in region (4)). The TNF processed in this manner now binds preferably to cell TNF receptors, since these, as homomultimeric molecules, have a considerably higher affinity than the monomeric, soluble receptor fragments. The selectivity of the TNF activity is therefore achieved with the selectokine according to the invention by means of two measures: on the one hand via the scFv-mediated selective enrichment of the inactive prodrug in the tumor and its retention even after proteolytic activation, and on the other hand via the site-specific conversion of the prodrug by proteases that can be detected in significant activity exclusively or preferentially in the tumor area. Moreover, a further preferential activity of the selectokine is achieved by the scFv-mediated binding of the TNF to membrane antigens, namely an improved biological activity compared with the conventionally used (soluble) TNF molecule, which activity is similar to that of the natural membrane-TNF molecule: due to the scFv-mediated fixing of the TNF, the dissociation equilibrium at the TNFR2 is shifted towards a more stable binding, and thus its activation is achieved. It is known that the simultaneous activation of both TNFR can lead to a cooperative signal mechanism and result in strengthened cellular reactions, in particular the activation of endothelial cells and the induction of apoptosis in tumor cells that in this respect are resistant to conventionally used (soluble) TNF.

Particularly preferred embodiments of the polypeptide according to the invention feature the amino acid sequences shown in FIG. 1 (SEQ ID NO 1) and 5 (SEQ ID NO 3).

A further subject of the present invention relates to a nucleic acid comprising a nucleotide sequence that codes for the polypeptide according to the invention. The term “nucleic acid” denotes a natural, semi-synthetic, synthetic, or modified nucleic acid molecule of deoxyribonucleotides, and/or ribonucleotides and/or modified nucleotides. Preferred embodiments of the nucleic acid according to the invention contain the nucleotide sequence shown in FIG. 1 (SEQ ID NO 2) and FIG. 5 (SEQ ID NO 4).

Furthermore a vector containing the above-defined nucleic acid is made available according to the invention. The vector is preferably capable of expression and/or amplification in a prokaryotic and/or eukaryotic cell. To this end, the vector preferably contains suitable regulatory elements such as promoters, enhancers, termination sequences, etc. The vector can also be used for the stable integration of the nucleic acid according to the invention into the genetic material of a host cell.

A further subject relates according to the invention to a host cell containing the above nucleic acid and/or the above vector. Suitable host cells, for example, are all mammalian cells, such as COS or CHO cells.

The present invention likewise makes available a method for the production of the polypeptide of the invention, comprising the steps

(a) cultivation of the above host cell in a culture medium under suitable conditions and

(b) isolation of the polypeptide of the invention from the host cells and/or the culture medium.

The polypeptide according to the invention is preferably produced by expression with the aid of suitable expression systems, preferably as secreted product of selectable, stable transfectants of the cell line CHO DG44 or after transient expression in COS7 cells. Other eukaryotic expression systems corresponding to the state of the art are e.g. Pichia pastoris, insect or mammalian cells, with the expression vectors for secretion suited to the respective cell system, e.g. as described for mammalian and insect cells in Brocks et al. (Immunotechnology 3:173-184, 1997). pPICZalpha vectors (INVITROGEN) are likewise suitable for expression and secretion in the yeast Pichia pastoris.

The polypeptide according to the invention, the nucleic acid, and/or the vector can be used advantageously for the production of pharmaceutical compositions for the treatment of pathological disorders.

A further development of the present invention therefore relates to a pharmaceutical composition containing a pharmaceutically effective amount of the polypeptide according to the invention and/or of the nucleic acid according to the invention and/or of the vector according to the invention, optionally combined with one or more pharmaceutically acceptable auxiliary agents, diluents, and/or carriers. The pharmaceutical composition is preferably used for the therapeutic treatment of carcinoses and/or infectious diseases and/or metabolic diseases. Particularly preferred fields of application of the pharmaceutical composition are the treatment of solid tumors as well as angiogenesis in pathological lesions. The pharmaceutical composition according to the invention can take any form acknowledged to be suitable in this professional field. It is preferably solid, liquid, or in the form of an aerosol.

The present invention thus likewise comprises a treatment method that includes the administration of a therapeutically adequate amount of the pharmaceutical composition according to the invention to a patient in need of the treatment. Suitable modes of administration of the pharmaceutical composition are known to those skilled in the art and comprise for example oral, intravenous, intra-arterial, intramuscular, nasal, rectal, and topical application. An intravenous administration can be carried out e.g. in the form of a bolus injection with subsequent injection intervals and/or in the form of an infusion. Both human and animal patients can be treated with the pharmaceutical composition of the present invention. The treatment method is preferably used for patients with the above-mentioned diseases.

### THE FIGURES SHOW

FIG. 1 shows the amino acid sequence (SEQ ID NO 1, top) and the corresponding cDNA nucleotide sequence (SEQ ID NO 2, bottom) of the selectokine prodrug W24 according to the invention.

FIG. 2 shows photographic representations of a Coomassie-stained SDS-PAGE gel and of the corresponding Western blot after incubation with anti-c-myc-mAK 9E10. The prodrug W24 was expressed in CHO-DG44 cells and purified by means of IMAC. The purified protein was applied under reducing (red.) and also non-reducing conditions.

FIG. 3 is a photographic representation of a Western blot analysis of a 12% SDS gel after detection with anti-c-myc-mAK 9E10. Track 1: purified prodrug W24 after incubation with PBS. Track 2: purified prodrug W24 after incubation with PBS plus tPA.

FIG. 4 is a graphic representation of the results of an apoptosis induction test on Kym1 cells with trypsin-activated prodrug W24 (▪) or non-activated prodrug W24 (▾).

FIG. 5 shows the amino acid sequence (SEQ ID NO 3, top) and the corresponding cDNA nucleotide sequence (SEQ ID NO 4, bottom) of the selectokine prodrug W33 according to the invention.

FIG. 6 (A) shows photographic representations of a Coomassie-stained SDS-PAGE gel and the corresponding Western blot after incubation with anti-c-myc-mAK 9E10. The prodrug W32 was expressed in CHO-DG44 cells and purified by means of IMAC. The purified protein was applied under reducing and also non-reducing conditions. (B) is a graphic representation of the results for determining the K_{D, app.} of the prodrug W32 with respect to FAP binding by means of FACS analysis. FAP-positive HT1080#33 cells (∘) and FAP-negative HT 1080 control cells (●) were incubated with serial dilutions of prodrug W32 and the cell-bound moiety was detected by means of indirect immunofluorescence intensity. The prodrug concentration used is shown versus the median fluorescence intensity (MFI).

FIG. 7 (A) is a graphic representation of the results of an apoptosis induction test on Kym1 cells with non-activated prodrug W32 (▪), trypsin-activated prodrug W32 (□), or wild type TNF (●). A representative of three experiments is shown. The photographic representation of a Coomassie-stained SDS-PAGE gel under reducing conditions of IMAC-purified prodrug W32 (left track) and of the IMAC-purified prodrug W32 after trypsin activation (right track) is inserted. The arrow corresponds to the expected MW of the activated prodrug W32. (B) is a graphic representation of the results of an apoptosis induction test on Kym1 cells in co-culture with prodrug-presenting, FAP-positive cells (HT1080#33) as well as with FAP-negative control cells (HT1080). HT1080+non-activated prodrug W32 (Δ), HT1080+trypsin-activated prodrug W32 (□), HT1080#33+non-activated prodrug W32 (▴), HT1080#33+trypsin-activated prodrug W32 (▪). (C) is a graphic representation of an experiment corresponding to that shown in (B), but the trypsin activation took place only after the binding to the HT cells and subsequent fixing. HT1080+non-activated prodrug W32 (∘), HT1080+trypsin-activated prodrug W32 (□), HT1080#33+non-activated prodrug W32 (●), HT1080#33+trypsin-activated prodrug W32 (▪). In each of the graphic representations of (B) and (C), a representative of three experiments is shown.

The present invention is explained in more detail with reference to the following non-limiting examples.

### EXAMPLES

### Example 1

Examples of the Sequence of TNF Selectokines

Examples of the sequence of a TNF selectokine with multiple cleavage sites in the linker and various receptor fragments:

1. scFv−TD_{tenascin}−hu TNF (AA1-157)−linker−huTNFR1 (AA 1-190).

2. scFv−TD_{tenascin}−hu TNF (AA1-157)−linker−huTNFR1 (M 60-120).

In another variant, potential endogenous cleavage sites in the huTNF molecule are removed by amino acid exchange (TNFmut 183F, R131Q) while maintaining the scFV, linker sequence, and receptor sequence as shown above by way of example.

As the trimerization domain, the coiled coil domain of tenascin-C (AA 110-139), which is highly conserved in various species, is used: Example of the AA sequence:

| | | |
|---|---|---|
| Chicken: | ACGCAAAPIVKDLLSRLEELEGLVSSLREQ | (Swissprot #P10039, SEQ ID NO 5)* |
| Human: | ACGCAAAPDVKELLSRLEELENLVSSLREQ | (Swissprot #P24821, SEQ ID NO 6)# |

* Nies, D. E., Hemesath, T. J., Kim, J. H., Guicher, J. R. and Stefansson, K. The complete cDNA sequence of human hexabrachion (Tenascin). A multidomain protein containing unique epidermal growth factor repeats. J. Biol. Chem. 266 (5), 2818-2823 (1991)

# Spring, J., Beck, K. and Chiquet-Ehrismann, R. Two contrary functions of tenascin: dissection of the active sites by recombinant tenascin fragments. Cell 59 (2), 325-334 (1989)

### Example 2

Linker Sequence

A processing sequence according to the invention is for example a linker with the protease cleavage sites for thrombin, tPA, Factor VIIa, and uPA (bottom amino acid sequence, SEQ ID NO 7; top cDNA nucleotide sequence, SEQ ID NO 8):

```
TCCGGAATGTACCCCAGAGGATCGATCGGCGCCCCCTTCGGCCGCGGCGCCCCCTTCGTACGCATC
  S G M Y P R G S I G A P F G R G A P F V R I
                      | Thrombin | | tPA | |FactorVIIa|
GAGGGTCGGGTC
  E G R V
| uPA |
```

### Exampl 3

Expr ssion, Purification, and Functi nal Charact rization of th TNF Selectokin Prodrug W24

Prodrug W24

The TNF selectokine prodrug W24 consists of the following components (from N- to C-terminus, amino acid residues (AA) are relative to SEQ ID NO 1):

1. AA 1-19: Leader peptide sequence

2. AA 20-285: scFv OS4 (specificity: human FAP; cf. Mersmann (2000) Dissertation Universitat Stuttgart [University of Stuttgart], Verlag Grauer, Stuttgart, ISBN 3-86186-335-9; Rippmann 1999, Dissertation Universitat Stuttgart, ISBN 3-86186-281-6)

3. AA 286-315: Trimerization domain of tenascin (chicken, see above) AA316-321: Linker

4. M 322486: Mutated form of the natural, human TNF precursor protein (26 kDa membrane form, Swissprot #P01375, 233 M) with deletions of the N-terminal 56 AA and of AA 78-89 (TNF_{Δ1-56, 78-89}), i.e. deletion of the cytoplasmic domain, the transmembrane domain, and the TACE cleavage site of the TNF precursor polypeptide

5. AA 487-512: Linker with protease cleavage sites (cf. Example 2)

6. AA 513-639: Human TNFR1 fragment containing the extracellular domains 1-3 (Swissprot #P19438, M 12-138; cf. Himmler et al. (1990) DNA and Cell Biology 9, 705-715)

7. AA 640-652 myc tag

8. M 653-658 His tag

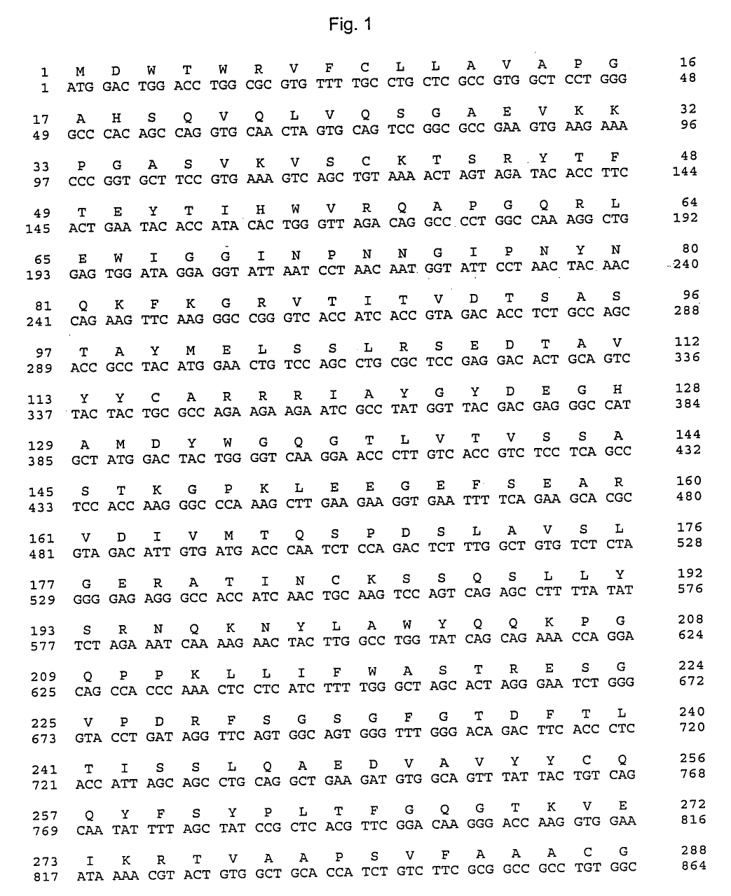

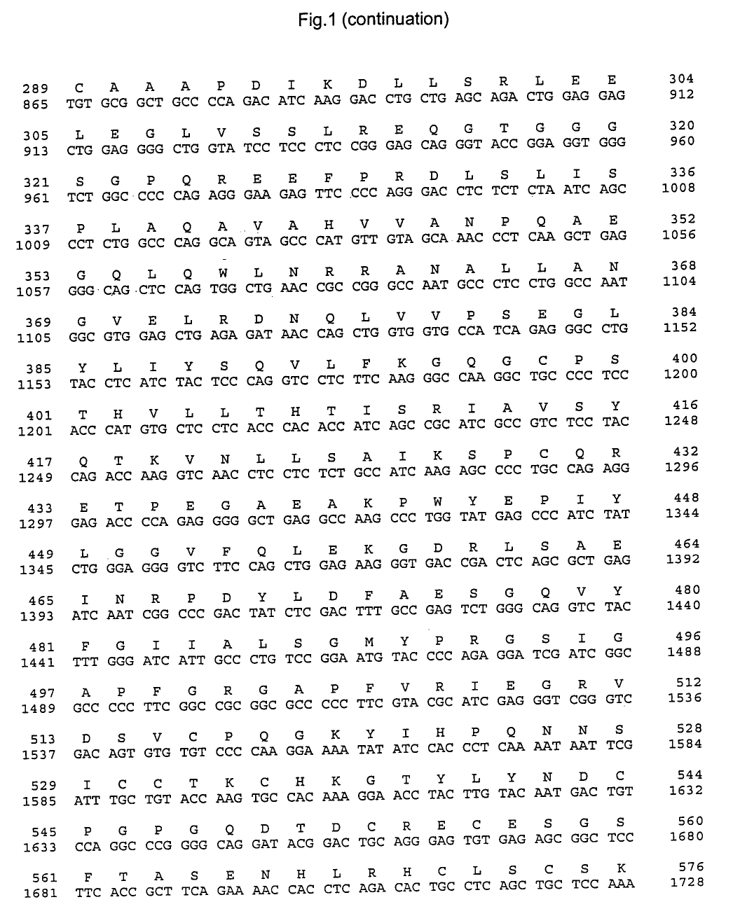

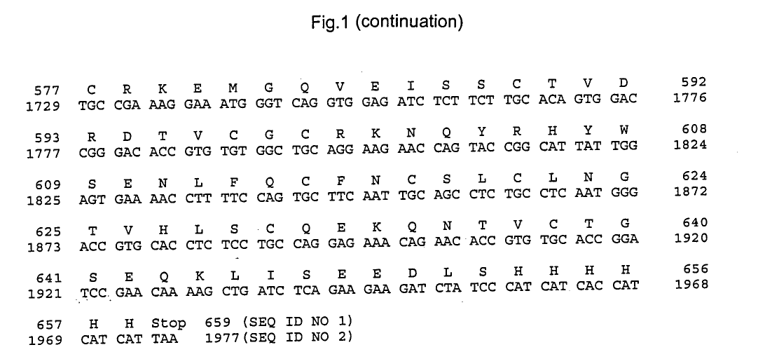

The amino acid sequence (SEQ ID NO 1) and the corresponding coding DNA sequence (SEQ ID NO 2) are shown in FIG. 1. The calculated MW of the protein moiety is 70.3 kDa.

Expression and Purification

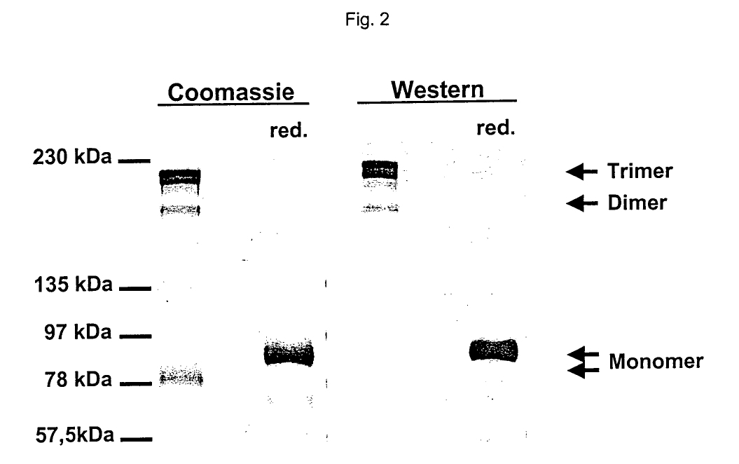

Prodrug W24 was purified from CHO supernatant by means of IMAC according to the instructions of the manufacturer (Pharmacia). In a Coomassie-stained SDS-PAGE gel, 400 ng (20 μL) of this was applied under reducing and non-reducing conditions, 2 μL was used in the Western blot with an anti-c-myc-mAk 9E10; cf. FIG. 2. The expression of the monomeric, dimeric, and trimeric construct is detected.

Cleavage of Prodrug W24 by tPA

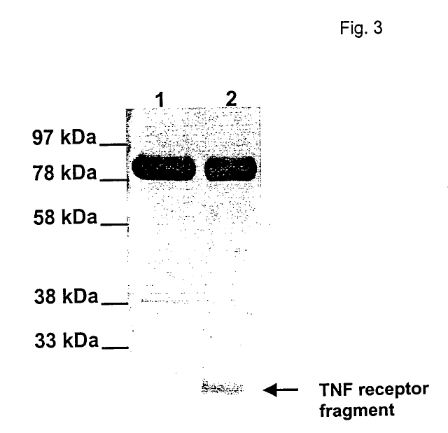

The purified prodrug W24 (600 ng) was incubated in PBS (50 μL) or in PBS+tPA (5 μg tPA in 50 μL PBS) at 37° C. for 16 h. After 12% SDS-PAGE (reducing) and Western blot, detection was carried out with anti-c-myc-mAk 9E10, followed by alkaline phosphatase-conjugated goat anti-mouse IgG serum. The appearance of a band below 33 kDa at the level of the expected size of the cleaved-off TNFR fragment (about 17 kDa), the C-terminus of which carries a myc tag, in the batch with tPA shows the partial digestion of prodrug W24; cf. FIG. 3. The activated TNF selectokine cannot be shown with this detection method.

Proteolytic Activation of Prodrug W24 by Trypsin

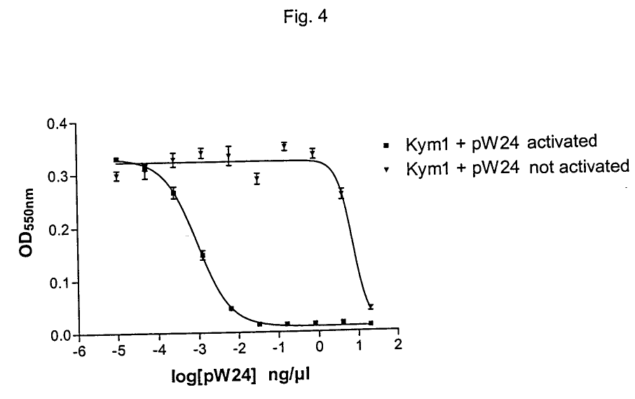

20,000 Kym1 cells in 50 μL standard culture medium (10% FCS) were sown the previous day in plates with 96 wells (4-fold values), and the following day 50 μL prodrug W24 dilution was added. The trypsin activation of IMAC-purified prodrug W24 (2 μg) took place in a total volume of 50 μL PBS with a final concentration of 100 μg/mL trypsin. The reaction was stopped after 5 min incubation at room temperature with 200 μL RPMI/10% FCS. The non-activated sample was treated identically, but without trypsin. The vitality was determined after 16 h by MTT staining. The results show (FIG. 4) that the non-processed TNF selectokine has no bioactivity (apoptosis induction) up to a concentration of about 2-3 μg/mL, while the selectokine activated by trypsin has a high specific bioactivity comparable to wild type TNF (LD₅₀=0.5 ng/mL). Determination of the LD₅₀ shows an approximately 4,000-fold increase in activity due to the processing.

### Exampl 4

Prodrug W33

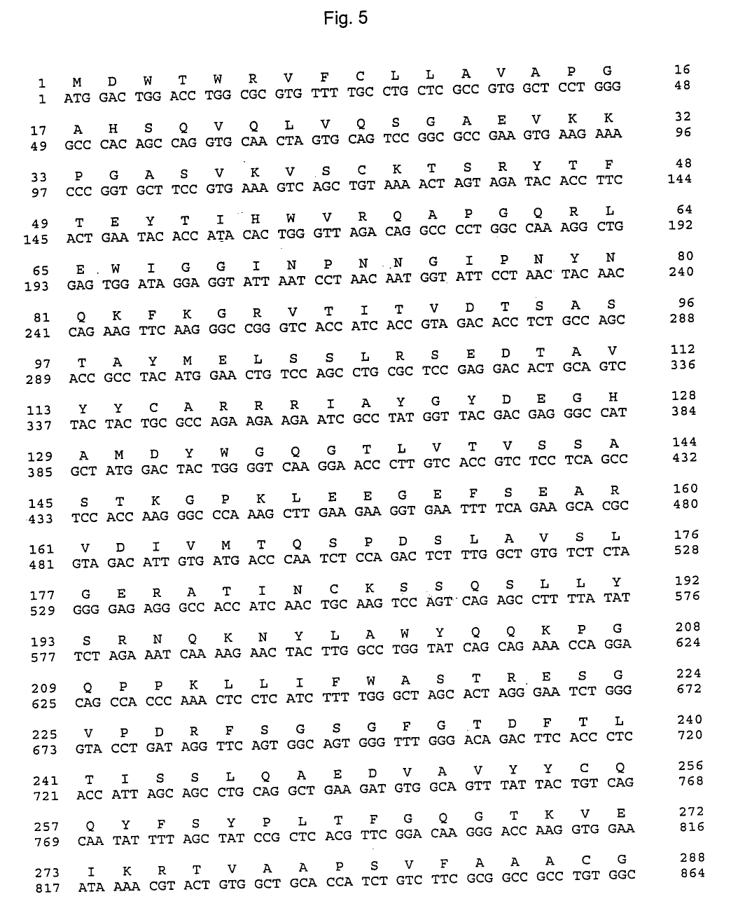

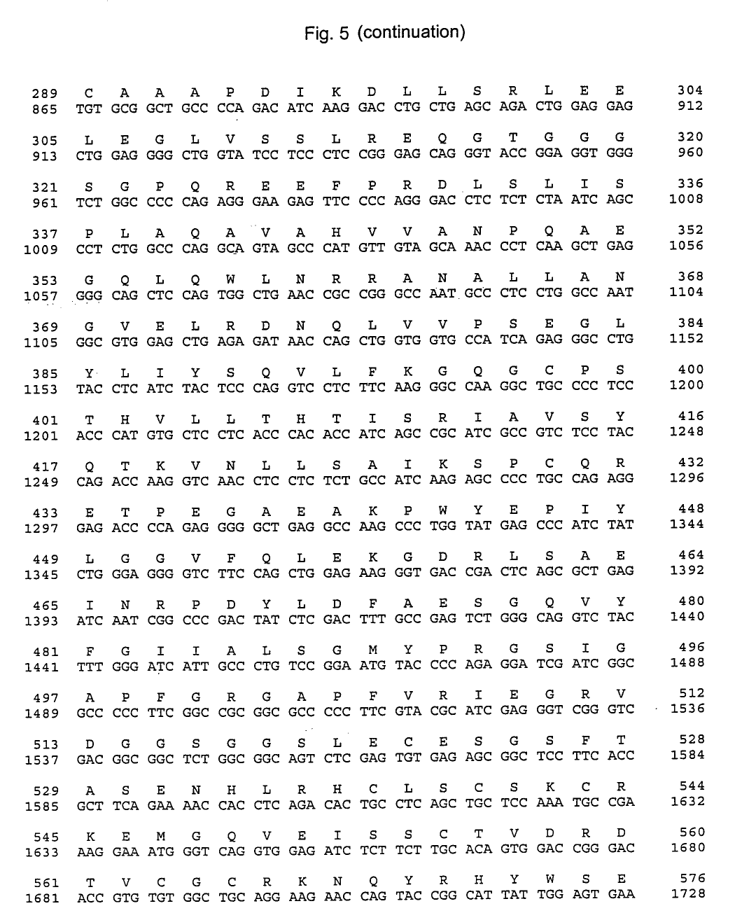

The prodrug W33 construct has the same functional properties as the prodrug W24 of Example 3, but differs from it by a longer protease-sensitive linker (AA 487-520) and a shorter TNFR fragment (AA 521-582; Swissprot #P19438, AA 54-115 of the human TNFR1; cf. Himmler et al. (1990) DNA and Cell Biology 9, 705-715). The amino acid sequence (SEQ ID NO 3) and the coding cDNA sequence (SEQ ID NO 4) of prodrug W33 are shown in FIG. 5.

### Example 5

Expression, Purification, and Functional Characterization of TNF Selectokine Prodrug W32

Prodrug W32

Another construct (prodrug W32) was produced that corresponds functionally to the prodrug W24 of Example 3, but contains as targeting module (1) a different antibody fragment (scFv MO36), which was isolated independently from a murine Ig gene library and was selected for human/murine FAP cross reactivity. In contrast, the targeting specificity of prodrug W24 of Example 3 is based on scFv OS4, which exclusively recognizes human FAP.

Biochemical Characterization and Antigen Binding Activity of TNF Selectokine Prodrug W32

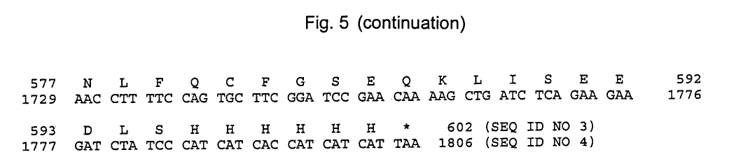

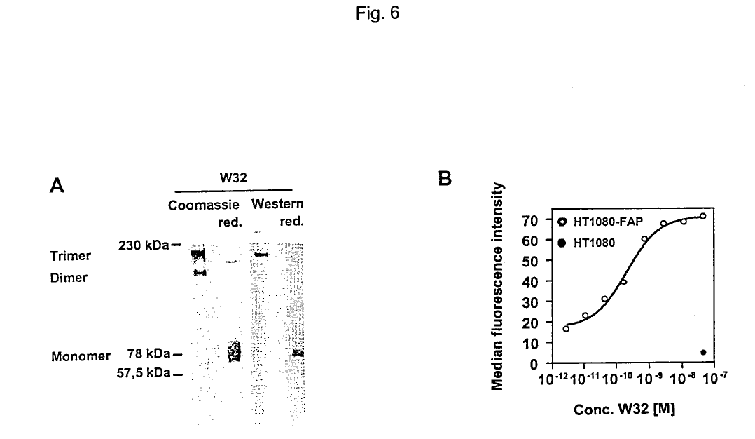

Prodrug W32 was expressed like construct W24 (Example 3), purified by means of IMAC, and analyzed by SDS-PAGE/Western blot; cf. FIG. 6A.

Moreover the K_{D, app.} of prodrug W32 for FAP binding was determined by means of FACS analysis; cf. FIG. 6B. The K_{D, app.} was calculated from the concentration at which the half-maximum signal was obtained. This gave a value of 2.4×10⁻¹⁰ M.

TNF Activity of Prodrug W32

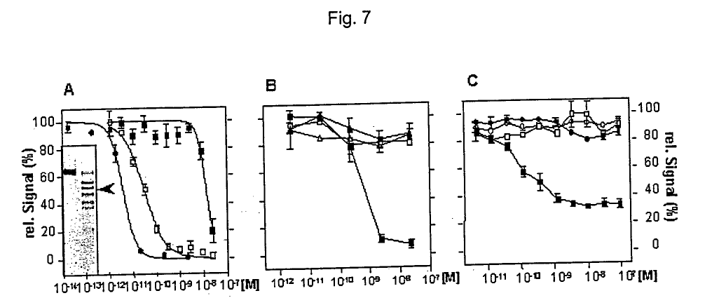

In the Kym-1 apoptosis test (cf. Example 3 for the procedure), the activated prodrug W32 has an activity comparable to naturally occurring TNF, whereas the non-processed construct develops apoptotic activity only at very much higher concentrations; cf. FIG. 7A.

Moreover, a juxtatropic apoptosis induction of the activated prodrug W32 was studied in co-culture with prodrug-presenting cells. For this, FAP-negative HT1080 control cells or FAP-positive HT1080#33 cells were incubated with serial dilutions of the prodrug or the trypsin-activated prodrug, washed, fixed, co-cultured with Kym-1, and the vitality of the cells was determined after 16 h; cf. FIG. 7B. In a further experiment with otherwise similar batches, the trypsin activation of the prodrug took place only after the binding to the cells and subsequent fixing of the cells; cf. FIG. 7C. In both cases a significant juxtatropic apoptosis induction by the activated prodrug is found.

### Example 6

Construction of the Polypeptides Selectokine W24 and W33 According to the Invention

The fusion proteins are produced as follows:

1. The single-chain antibody fragment (scFv) OS4 (referred to below as OS4) is the version of the FAP-specific mAb F19 humanized by CDR grafting (Rettig et al. 1988) and described in Rippmann J. F. (Dissertation Univ. Stuttgart, Verlag Grauer, Stuttgart, 1999) as well as Rippmann et al. (Appl EnvMicrobiol 64:4862-4869, 1998).

2. In the eukaryotic expression vector pG1D105 (described in EP 0 953 639) with the expression cassette for the heavy immunoglobulin chain of mAb F19, the latter was exchanged by means of BstE2 and Nael for the minibody OS4 cassette consisting of scFv OS4; hinge-CH3 region of an hulgG1; myc/his tag of plasmid pW7 (described in Wuest, T., Dissertation Univ. Stuttgart, 2001). The Fc fragment (hinge region and CH3 region of hu IgGI) is removed selectively by an Notl/BamH1 digestion.

3. The CDNA sequence of the trimerization domain (e.g. AA 110-139 of chicken tenascin) was amplified by proof-reading PCR by means of primer 1 (SEQ ID NO 10) and 2 (SEQ ID NO 11), as a result of which the cleavage sites Not1 and Kpn1 were introduced. The human TNF fragment was amplified by means of primer 3 (SEQ ID NO 12) and 4 (SEQ ID NO 13) from a non-cleavable membranous TNF mutant (membrane-TNF, TNFdelta1-12, Grell et al., Cell 83:793-802, 1995), as a result of which the cleavage site Kpn1 was introduced at the 5′-end, the cleavage sites Acc3 and BamH1 were introduced at the 3′-end, and a sequence coding for the peptide linker TyrGlyGlyGlySer (SEQ ID NO 9) was introduced between the sequence segments coding for the tenascin and TNF domains. The two fragments were inserted into the Notl/BamH1-digested cloning intermediate described under 2., using the Not1, Kpn1, and BamH1 cleavage sites.

4. The cloning of the protease-sensitive linker and the human TNF receptor 1 fragment took place via several intermediates. For this, the TNF receptor 1 fragment (cysteine-rich domains 1-3; AA 12-138; Swissprot #10039) was PCR-amplified with primers 5 (SEQ ID NO 14) and 6 (SEQ ID NO 15) from the plasmid pADBTNF-R (Himmler et al. DNA Cell Biol 9:705-715, 1990), and reamplified with primers 7 (SEQ ID NO 16) and 6 (SEQ ID NO 15), as a result of which the cleavage sites Acc3, BamH1 and a linker 5′ of the TNFR fragment was[sic] introduced that codes for the protease cleavage sites. This fragment was cloned via cleavage sites Acc3/BamH1 into the intermediate described in 3.

5. A modification of the protease-sensitive linker in the linker TNFR1 fragment was introduced via PCR amplification with primers 8 (SEQ ID NO 17) and 6 (SEQ ID NO 15). The fragment thus obtained was inserted via cleavage sites Clal and BamH1 into the intermediate described under 4., replacing the previously obtained linker TNFR fragment. The eukaryotic expression plasmid pW24 produced in this manner allows the expression of the TNF selectokine prodrug W24.

6. In a further embodiment of the TNF selectokine prodrug, a TNF receptor is PCR-amplified with primers 9 and 10, resulting in a sequence with a 5′ linker coding for AA 54-115 of human TNFR1. This fragment is inserted into pW24 via the Sall and BamH1 cleavage sites and replaces the linker TNFR1 fragment contained there. The resulting expression plasmid pW33 allows the expression of the TNF selectokine prod rug W33.

7. To obtain a TNF selectokine prodrug (W24 and W33), CHO-DG44 cells with the constructs described under 5. and 6. were transfected with lipofectamine (Gibco-BRL) according to the manufacturer's instructions and subsequently selected by means of hypoxanthine and thymidine-free (HT⁻) CHO—S—SFM medium (Life Technologies) for stable integration of the constructs into the genome. Increased expression was obtained by a stepwise increase of the selection reagent methotrexate (0.1; 1; 10 μM). Both W24 and W33 were purified from the culture supernatants under sterile conditions by means of immobilized metal affinity chromatography (IMAC) as described in Rippmann et al. (Appl EnvMicrobiol 64:4862-4869, 1998) and were stored at 4° C. for further use.

All cloning and PCR amplification steps were performed by customary standard procedures with the following primers. All constructs were sequenced to verify their cDNA sequence. The relevant cleavage sites are printed in bold.

Primer 1

(SEQ ID NO 10)

Not1
5′ TAA ATA GGG GCC CAC AGC CAG GCG GCC GCC TGT GGC TGT GCG GCT GC 3′
Primer 2

(SEQ ID 11)

Kpn1
5′ ATA AAT GGT ACC CTG CTC CCG GAG GGA GGA 3′
Primer 3

(SEQ ID NO 12)

Kpn1
5′ GAG AGG GTA CCG GAG GTG GGT CTG GCC CCC AGA GGG AAG AG 3′
Primer 4

(SEQ ID NO 13)

BamH1 Acc3
5′ TTG TTC GGA TCC ACG ACC CTC GAT TCC GGA CAG GGC AAT GAT CCC AAAG 3′
Primer 5

(SEQ ID NO 14)

BamH1
5′ CTC GGG ATC CGG CGG TGG CAG ATC TGG CGG GGG TGG GGT CGA CAG TGT GTG TCC CCA AGG 3′
Primer 6

(SEQ ID NO 15)

BamH1
5′ CCT GCG GAT CCG GTG CAC ACG GTG TTC TG 3′
Primer 7

(SEQ ID NO 16)

Acc3 Cla1
5′ GCC TTC CGG AAT GTA CCC CAG AGG ATC GAT TGG TGG CAG ATC TGG CGG 3′
Primer 8

(SEQ ID NO 17)

Cla1
5′ AGT GGA TCG ATC GGC GCC CCC TTC GGC CGC GGC GCC CCC TTC GTA CGC ATC GAG GGT CGG GTC GAC AGT GTG
TGT C 3′

1. Polypeptide with an amino acid sequence, comprising from the N- to the C-terminus
(1) a region that selectively recognizes a specific macromolecule on a cell surface and/or a component of the extracellular matrix,
(2) a region that comprises a peptide linker,
(3) a region with a biological activity for a specific target molecule,
(4) a region that features at least one processing site, and
(5) a region that inhibits the biological activity of region (3) by intramolecular binding and/or interaction,
wherein the biological activity of region (3) can be released by the processing in vivo of the at least one processing site in region (4).

2. Polypeptide according to claim 1, wherein region (3) contains an amino acid sequence of a cytokine or a fragment thereof.

3. Polypeptide according to claim 2, wherein the cytokine is a tumor necrosis factor (TNF), or a biologically active derivative or biologically active mutant thereof.

4. Polypeptide according to any one of claims 1 to 3, wherein the specific target molecule of region (3) is a cell membrane-bound cytokine receptor.

5. Polypeptide according to claim 3 or 4, wherein region (3) comprises amino acid residues 322 to 486 of SEQ ID NO 1.

6. Polypeptide according to one of claims 1 to 5, wherein the at least one processing site in region (4) is a cleavage site for a protease.

7. Polypeptide according to claim 5, wherein the protease is a urokinase-type plasminogen activator (uPA), tissue plasminogen activator (tPA), activated coagulation Factor VIIa, a matrix metalloprotease, or an FAP protease.

8. Polypeptide according to claim 6 or 7, wherein region (4) comprises amino acid residues 487 to 512 of SEQ ID NO 1 or amino acid residues 487 to 520 of SEQ ID NO 3.

9. Polypeptide according to one of claims 1 to 8, wherein region (5) has at least one binding site for region (3).

10. Polypeptide according to claim 9, wherein region (5) is a receptor for a cytokine or a fragment thereof.

11. Polypeptide according to claim 10, wherein the receptor comprises the complete or partial extracellular domain of a human TNF receptor and/or a TNF-binding virus protein or a mutant thereof or a synthetic TNF-binding compound.

12. Polypeptide according to claim 11, wherein region (5) comprises amino acid residues 513 to 639 of SEQ ID NO 1 or amino acid residues 521 to 582 of SEQ ID NO 3.

13. Polypeptide according to one of claims 1 to 12, wherein the peptide linker in region (2) is a trimerization module and connects region (1) to region (3).

14. Polypeptide according to claim 13, wherein the trimerization module comprises a naturally occurring or synthetic peptide with intrinsic trimerization properties.

15. Polypeptide according to claim 14, wherein region (2) comprises the amino acid sequence of SEQ ID NO 5 or SEQ ID NO 6.

16. Polypeptide according to any one of claims 1 to 15, wherein region (1) is specific for a cell surface molecule that is expressed in tumor lesions and/or proliferating endothelial cells associated with the process of angiogenesis.

17. Polypeptide according to any one of claims 1 to 15, wherein region (1) is specific for a component of the extracellular matrix present in tumor lesions and/or angiogenesis areas of pathological lesions.

18. Polypeptide according to any one of claims 1 to 15, wherein region (1) is specific for a component of the malignant tumor cell itself.

19. Polypeptide according to one of claims 1 to 18, wherein region (1) is a murine, humanized, or human antibody or a fragment thereof with defined antigen specificity.

20. Polypeptide according to claim 19, wherein the antibody fragment is an scFv or Fab fragment.

21. Polypeptide according to claim 20, wherein region (1) comprises amino acid residues 20 to 285 of SEQ ID NO 1.

22. Nucleic acid that comprises a nucleotide sequence coding for the polypeptide according to any one of claims 1 to 21.

23. Vector containing the nucleic acid according to claim 22.

24. Vector according to claim 23 that is capable of expression and/or amplification in a prokaryotic and/or eukaryotic cell.

25. Host cell containing the nucleic acid according to claim 22 and/or the vector according to claim 23 or 24.

26. Method for the production of the polypeptide according to one of claims 1 to 21, comprising the steps
(a) cultivation of the host cell according to claim 25 in a culture medium under suitable conditions and
(b) isolation of the polypeptide according to one of claims 1 to 21 from the host cells and/or the culture medium.

27. Pharmaceutical composition containing a pharmaceutically effective amount of the polypeptide according to any one of claims 1 to 21 and/or of the nucleic acid according to claim 22 and/or of the vector according to claim 23 or 24, optionally combined with one or more pharmaceutically acceptable auxiliary agents, diluents, and/or carriers.

28. Pharmaceutical composition according to claim 27 for the therapeutic treatment of solid tumors and/or of angiogenesis in pathological lesions.

29. Pharmaceutical composition according to claim 27 or 28 that is solid, liquid, or in the form of an aerosol.


SEQUENCE LISTING (18 sequences)

SEQ ID NO: 1 - protein, 658 aa, Artificial Sequence
  Description of the artificial sequence selectokine W24 amino acid sequence
MDWTWRVFCLLAVAPGAHSQVQLVQSGAEVKKPGASVKVSCKTSRYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSSASTKGPKLEEGEFSEARVDIVMTQSPDSLAVSLGERATINCKSSQSLLYSRNQKNYLAWYQQKPGQPPKLLIFWASTRESGVPDRFSGSGFGTDFTLTISSLQAEDVAVYYCQQYFSYPLTFGQGTKVEIKRTVAAPSVFAAACGCAAAPDIKDLLSRLEELEGLVSSLREQGTGGGSGPQREEFPRDLSLISPLAQAVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALSGMYPRGSIGAPFGRGAPFVRIEGRVDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTGSEQKLISEEDLSHHHHHH

SEQ ID NO: 2 - DNA, 1977 bp, Artificial Sequence
  Description of the artificial sequence selectokine W24 cDNA sequence
atggactggacctggcgcgtgttttgcctgctcgccgtggctcctggggcccacagccaggtgcaactagtgcagtccggcgccgaagtgaagaaacccggtgcttccgtgaaagtcagctgtaaaactagtagatacaccttcactgaatacaccatacactgggttagacaggcccctggccaaaggctggagtggataggaggtattaatcctaacaatggtattcctaactacaaccagaagttcaagggccgggtcaccatcaccgtagacacctctgccagcaccgcctacatggaactgtccagcctgcgctccgaggacactgcagtctactactgcgccagaagaagaatcgcctatggttacgacgagggccatgctatggactactggggtcaaggaacccttgtcaccgtctcctcagcctccaccaagggcccaaagcttgaagaaggtgaattttcagaagcacgcgtagacattgtgatgacccaatctccagactctttggctgtgtctctaggggagagggccaccatcaactgcaagtccagtcagagccttttatattctagaaatcaaaagaactacttggcctggtatcagcagaaaccaggacagccacccaaactcctcatcttttgggctagcactagggaatctggggtacctgataggttcagtggcagtgggtttgggacagacttcaccctcaccattagcagcctgcaggctgaagatgtggcagtttattactgtcagcaatattttagctatccgctcacgttcggacaagggaccaaggtggaaataaaacgtactgtggctgcaccatctgtcttcgcggccgcctgtggctgtgcggctgccccagacatcaaggacctgctgagcagactggaggagctggaggggctggtatcctccctccgggagcagggtaccggaggtgggtctggcccccagagggaagagttccccagggacctctctctaatcagccctctggcccaggcagtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtccggaatgtaccccagaggatcgatcggcgcccccttcggccgcggcgcccccttcgtacgcatcgagggtcgggtcgacagtgtgtgtccccaaggaaaatatatccaccctcaaaataattcgatttgctgtaccaagtgccacaaaggaacctacttgtacaatgactgtccaggcccggggcaggatacggactgcagggagtgtgagagcggctccttcaccgcttcagaaaaccacctcagacactgcctcagctgctccaaatgccgaaaggaaatgggtcaggtggagatctcttcttgcacagtggaccgggacaccgtgtgtggctgcaggaagaaccagtaccggcattattggagtgaaaaccttttccagtgcttcaattgcagcctctgcctcaatgggaccgtgcacctctcctgccaggagaaacagaacaccgtgtgcaccggatccgaacaaaagctgatctcagaagaagatctatcccatcatcaccatcatcattaa

SEQ ID NO: 3 - protein, 601 aa, Artificial Sequence
  Description of the artificial sequence selectokine W33 amino acid sequence
MDWTWRVFCLLAVAPGAHSQVQLVQSGAEVKKPGASVKVSCKTSRYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSSASTKGPKLEEGEFSEARVDIVMTQSPDSLAVSLGERATINCKSSQSLLYSRNQKNYLAWYQQKPGQPPKLLIFWASTRESGVPDRFSGSGFGTDFTLTISSLQAEDVAVYYCQQYFSYPLTFGQGTKVEIKRTVAAPSVFAAACGCAAAPDIKDLLSRLEELEGLVSSLREQGTGGGSGPQREEFPRDLSLISPLAQAVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALSGMYPRGSIGAPFGRGAPFVRIEGRVDGGSGGSLECESGSFTASENHLRHCLSCSKCRKEMGQVEISSCTVDRDTVCGCRKNQYRHYWSENLFQCFGSEQKLISEEDLSHHHHHH

SEQ ID NO: 4 - DNA, 1806 bp, Artificial Sequence
  Description of the artificial sequence selectokine W33 cDNA sequence
atggactggacctggcgcgtgttttgcctgctcgccgtggctcctggggcccacagccaggtgcaactagtgcagtccggcgccgaagtgaagaaacccggtgcttccgtgaaagtcagctgtaaaactagtagatacaccttcactgaatacaccatacactgggttagacaggcccctggccaaaggctggagtggataggaggtattaatcctaacaatggtattcctaactacaaccagaagttcaagggccgggtcaccatcaccgtagacacctctgccagcaccgcctacatggaactgtccagcctgcgctccgaggacactgcagtctactactgcgccagaagaagaatcgcctatggttacgacgagggccatgctatggactactggggtcaaggaacccttgtcaccgtctcctcagcctccaccaagggcccaaagcttgaagaaggtgaattttcagaagcacgcgtagacattgtgatgacccaatctccagactctttggctgtgtctctaggggagagggccaccatcaactgcaagtccagtcagagccttttatattctagaaatcaaaagaactacttggcctggtatcagcagaaaccaggacagccacccaaactcctcatcttttgggctagcactagggaatctggggtacctgataggttcagtggcagtgggtttgggacagacttcaccctcaccattagcagcctgcaggctgaagatgtggcagtttattactgtcagcaatattttagctatccgctcacgttcggacaagggaccaaggtggaaataaaacgtactgtggctgcaccatctgtcttcgcggccgcctgtggctgtgcggctgccccagacatcaaggacctgctgagcagactggaggagctggaggggctggtatcctccctccgggagcagggtaccggaggtgggtctggcccccagagggaagagttccccagggacctctctctaatcagccctctggcccaggcagtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtccggaatgtaccccagaggatcgatcggcgcccccttcggccgcggcgcccccttcgtacgcatcgagggtcgggtcgacggcggctctggcggcagtctcgagtgtgagagcggctccttcaccgcttcagaaaaccacctcagacactgcctcagctgctccaaatgccgaaaggaaatgggtcaggtggagatctcttcttgcacagtggaccgggacaccgtgtgtggctgcaggaagaaccagtaccggcattattggagtgaaaaccttttccagtgcttcggatccgaacaaaagctgatctcagaagaagatctatcccatcatcaccatcatcattaa

SEQ ID NO: 5 - protein, 30 aa, Gallus gallus
ACGCAAAPIVKDLLSRLEELEGLVSSLREQ

SEQ ID NO: 6 - protein, 30 aa, Homo sapiens
ACGCAAAPDVKELLSRLEELENLVSSLREQ

SEQ ID NO: 7 - protein, 26 aa, Artificial Sequence
  Description of the artificial sequence For processing sequence with protease cleavage sites for cDNA coding for thrombin, tPA, Factor VIIa and uPA
SGMYPRGSIGAPFGRGAPFVRIEGRV

SEQ ID NO: 8 - DNA, 78 bp, Artificial Sequence
  Description of the artificial sequence Peptide linker
tccggaatgtaccccagaggatcgatcggcgcccccttcggccgcggcgcccccttcgtacgcatcgagggtcgggtc

SEQ ID NO: 9 - protein, 5 aa, Artificial Sequence
  Description of the artificial sequence Peptide Linker
YGGGS

SEQ ID NO: 10 - DNA, 47 bp, Artificial Sequence
  Description of the artificial sequence Primer 1 for the amplification of the trimerization of chicken tenascin
taaataggggcccacagccaggcggccgcctgtggctgtgcggctgc

SEQ ID NO: 11 - DNA, 30 bp, Artificial Sequence
  Description of the artificial sequence Primer 2 for the amplification of the trimerization domain of chicken tenascin
ataaatggtaccctgctcccggagggagga

SEQ ID NO: 12 - DNA, 41 bp, Artificial Sequence
  Description of the artificial sequence Primer 3 for the amplification of a human TNF fragment
gagagggtaccggaggtgggtctggcccccagagggaagag

SEQ ID NO: 13 - DNA, 49 bp, Artificial Sequence
  Description of the artificial sequence Primer 4 for the amplification of a human TNF fragment
ttgttcggatccacgaccctcgattccggacagggcaatgatcccaaag

SEQ ID NO: 14 - DNA, 60 bp, Artificial Sequence
  Description of the artificial sequence Primer 5 for the amplification of a TNFR1-Fragments
ctcgggatccggcggtggcagatctggcgggggtggggtcgacagtgtgtgtccccaagg

SEQ ID NO: 15 - DNA, 29 bp, Artificial Sequence
  Description of the artificial sequence Primer 6 for the amplification of a TNFR1-Fragments
cctgcggatccggtgcacacggtgttctg

SEQ ID NO: 16 - DNA, 48 bp, Artificial Sequence
  Description of the artificial sequence Primer 7 for the re-amplification of a TNFR1-Fragments
gccttccggaatgtaccccagaggatcgattggtggcagatctggcgg

SEQ ID NO: 17 - DNA, 75 bp, Artificial Sequence
  Description of the artificial sequence Primer 8 for the introduction of a protease-sensitive linker into the linker TNFR fragment by means of PCR amplification
agtggatcgatcggcgcccccttcggccgcggcgcccccttcgtacgcatcgagggtcgggtcgacagtgtgtgt

SEQ ID NO: 18 - protein, 8 aa, Artificial Sequence
  Description of the artificial sequence MMP-9 recognition sequence
GPLPVRGK